(19) United States
(12) Patent Application Publication
(10) Pub. No.: US 2013/0149696 A1
(43) Pub. Date: Jun. 13, 2013

(54) HIGH-THROUGHPUT METHOD FOR DETERMINING THE PRESENCE OF PAPILLOMAVIRUS-NEUTRALIZING ANTIBODIES IN A SAMPLE

(71) Applicants: Michael Pawlita, Eschelbronn (DE); Ivonne Rubio, Heidelberg (DE); Martin Müller, Neckargemund (DE); Peter Sehr, Heidelberg (DE)

(72) Inventors: Michael Pawlita, Eschelbronn (DE); Ivonne Rubio, Heidelberg (DE); Martin Müller, Neckargemund (DE); Peter Sehr, Heidelberg (DE)

(73) Assignees: European Molecular Biology Laboratory; Deutsches Krebsforschungszentrum

(21) Appl. No.: 13/700,672

(22) PCT Filed: May 31, 2011
(86) PCT No.: PCT/EP2011/058966

§ 371 Date: Feb. 13, 2013

(30) Foreign Application Priority Data
Jun. 2, 2010 (EP) 10164765.9

(51) Int. Cl. G01N 33/68

(52) U.S. Cl. CPC G01N 33/6854
USPC 435/5; 435/174; 435/287.2

(57) ABSTRACT

The present invention relates to a method for the determination of the presence of PV-neutralizing antibodies in a sample, comprising a) contacting said sample with infectious PV particles comprising a reporter gene, wherein the gene product of said reporter gene is secreted into the growth medium, b) contacting the PV particles from a) with host cells, and c) determining PV-neutralizing antibodies based on the amount of gene product from said reporter gene, wherein, preferably, a lower amount of said gene product as compared to a reference amount is indicative of the presence of PV-neutralizing antibodies. It further relates to a host cell strongly adhering to multi-cluster plates for use in a method for diagnosing anti-PV immunity comprising the method of the present invention.

The present invention relates to a method for the determination of the presence of papillomavirus (PV)-neutralizing antibodies in a sample, comprising a) contacting said sample with infectious PV particles comprising a reporter gene, wherein the gene product of said reporter gene is secreted into the growth medium, b) contacting the PV particles from a) with host cells, and c) determining PV-neutralizing antibodies based on the amount of gene product from said reporter gene. It further relates to a host cell strongly adhering to a solid support for use in a method for diagnosing anti-PV immunity.

Papillomaviruses (PV) infect the skin and mucosa, especially of the anogenital and respiratory tracts of humans and are thus divided into mucosal and skin types. Most PV types cause benign lesions like warts or papilloma; association of some types with various forms of malignancies has, however, been established. Accordingly, mucosal PV types are divided into low-risk types not associated with malignancies, high-risk types with an established association with cancer development, e.g. in cervical cancer, and putative high-risk types, where such an association is presumed.

Persistent infection with high-risk human papillomaviruses is a major risk factor for the development of cervical cancer and occurs in approximately 20% of infected women. Lesions resulting from persistent infection can progress to cervical intraepithelial grade I lesions (CIN1). 20% of these CIN1 lesions progress into CIN2 lesions, and some of these into CIN3 lesions and cervical cancer. The process of progression to CIN3 and cervical cancer is slow, often taking more than ten years after initial infection with the virus.

The late PV proteins L1 and L2 are structural proteins building up the viral capsid. The major capsid protein L1 is currently used in prophylactic vaccinations against HPV infection in humans. The formulations Cervarix™ and Gardasil® both contain virus-like particles (VLPs) consisting of L1 protein. Gardasil® includes VLPs of HPV types 6, 11, 16 and 18, which are expressed in and purified from S. cerevisiae. Cervarix™ consists of only HPV types 16 and 18. The VLPs for Cervarix™ are produced in insect cells infected with recombinant baculoviruses.

It has been shown that vaccination with the current formulations leads to the production of antibodies preventing PV from infecting body cells (neutralizing antibodies). It is, however, not clear if this is the case for each patient vaccinated; thus, it is desirable to have a method to detect neutralizing antibodies. Moreover, titers of anti-HPV antibodies have been shown to decrease with time after vaccination in humans, thus it is desirable to have a testing system available providing infounation as to when a booster vaccination should be applied. Since it is the neutralizing antibodies that confer protection against PV infection, such a testing system preferably provides information on titers of neutralizing antibodies.

Currently, two methods of detennining titers of neutralizing antibodies are available in the art. The first one uses antibody binding competition to determine the titer of neutralizing antibodies. In this system, a monoclonal antibody recognizing a distinct epitope on the surface of the HPV virion essential for infection is brought into competition with putative neutralizing antibodies. To the extent the monclonal antibody is prevented from binding, it is assumed that it was outcompeted by neutralizing antibodies. Such methods have been devised for HPV16 and HPV18; results with HPV18 are, however, conflicting in that so far no correlation between vaccination, infectibility, and measured titers of neutralizing antibodies were obtained.

The second method available is the pseudovirion-based neutralization assay (PBNA). In this method, PV-pseudovirions containing DNA coding for a reporter gene are incubated with the serum to be tested and then allowed to infect suitable host cells. Binding of neutralizing antibodies to the pseudovirions decreases infection of host cells, which in turn decreases expression of the reporter gene; this decrease of reporter gene expression can be measured by the appropriate methods. The downside of the PBNA is that the different steps have to be pipetted by hand because the assay is not suited for automated pipetting, which decreases reproducibility of results and poses severe limits on the number of samples that can be processed in parallel.

The technical problem underlying the present invention, thus, could be seen as the provision of means and methods for complying with the aforementioned needs. The said technical problem is solved by the embodiments characterized in the claims and herein below.

Accordingly, the current invention relates to a method for the determination of the presence of PV-neutralizing antibodies in a sample comprising a) contacting said sample with infectious PV particles comprising a reporter gene, wherein the gene product of said reporter gene is secreted into the growth medium, b) contacting the PV particles from a) with host cells, and c) determining the amount of gene product from said reporter gene, wherein a low amount of said gene product as compared to a control is indicative of the presence of PV-neutralizing antibodies.

As used herein, the term “papillomavirus” (PV) relates to a DNA virus from the papillomaviridae family of viruses that infects the skin and mucous membranes of mammals, preferably livestock, more preferably cattle and horses, most preferably humans.

Bovine papillomaviruses (BPV) are associated with several forms of cutaneous and mucosal papilloma in cattle. Based on sequence relatedness, BPV types 1 to 10 have been characterized so far. Additional candidate types, including bovine alimentary papillomavirus-11 (BaPV-11), BPV1bis, BPV3bis, and BPV-BAA5-Japan have been described. BPV infections are common in cattle, with around 50% of cattle being estimated to bear lesions/warts in the UK (Campo, M.S., 1995. Infection by bovine papillomavirus and prospects for vaccination. Trends in microbiology 3, 92-7).

For human PV (HPV), more than 110 HPV genotypes have been described (de Villiers, E. M., C. Fauquet, T. R. Broker, H. U. Bernard, and H. zur Hausen. 2004. Classification of papillomaviruses. Virology 324:17-27). Approximately 50 HPV genotypes are known to infect the mucosa. These mucosal genotypes are classified into three different groups based on their epidemiological association with cancer: “low-risk” human papillomaviruses (LR-HPV), “high-risk” human papillomaviruses (HR-HPV) and “putative high-risk” human papillomaviruses (pHR-HPV). It is also known that HR-HPVs can cause vulvar, anal, vaginal, penile, and oropharyngeal cancer, as well as vaginal intraepithelial neoplasia, anal intraepithelial neoplasia, vulvar intraepithelial neoplasia, and penile intraepithelial neoplasia.

Preferably, HPVs are mucosal HPVs; more preferably, HPVs of the current invention are High-risk HPV genotypes (HR-HPVs), which are the main cause for the development of cervical cancer, more preferably HPVs are HPV 31, 33, 35, 39, 45, 51, 52, 56, 58, 59, 68, 73 and 82, most preferably HPV16 or HPV18.

As used herein, the term “antibody” relates to a molecule from the subgroup of gamma globulin proteins which is also referred to as the immunoglobulins (Ig). Antibodies can, preferably, be of any subtype, i.e. IgA, IgD, IgE, IgM or, more preferably, IgG.

The term “PV-neutralizing antibody” relates to an antibody preventing PV from infecting host cells. Said inhibition preferably is caused by binding of the PV-neutralizing antibody to an epitope of a PV virion required for effective infection, preventing interaction of said epitope with components of the host cell from occurring. It is to be understood that neutralization as used herein does not necessarily mean the complete abolishment of infection in all cases. Inhibitory antibodies, preferably, reduce PV infection of host cells by at least 30%, at least 40%, or at least 50% as compared to a reference.

Preferably, determining the presence of PV-neutralizing antibodies referred to in this invention relates to determining the presence of said antibodies and/or to measuring the amount or concentration, preferably semi-quantitatively or quantitatively. Most preferably, the amount is measured as a titer, i.e. the maximum dilution of a sample that still affects a pre-determined degree of infection inhibition. Preferably, the determination includes a normalization step for the quantification of PV-neutralizing antibodies. Normalization and thus quantification is preferably achieved by adding a predefined amount of characterized PV-neutralizing antibodies to a reaction mixture. Said characterized PV-neutralizing antibodies, preferably, are antibodies where the amount required to achieve a certain level of neutralization has been pre-determined. The principle of the nounalization is to determine the amount or dilution of sample required to achieve the same level of neutralization, e.g. 50% inhibition of infection as compared to the inhibition by a pre-defined amount of characterized PV-neutralizing antibodies. For quantification, neutralization can be compared to a standard curve using characterized PV-neutralizing antibodies or to other suitable reference material. This can be done by the skilled person without further ado.

As used herein, the term “sample” relates to a sample of a body fluid, to a sample of separated cells or to a sample from a tissue or an organ or to a sample of wash/rinse fluid obtained from an outer or inner body surface. The sample comprises immunoglobulins from at least one class, preferably, the sample comprises IgG. Samples can be obtained by well known techniques and include, preferably, scrapes, swabs or biopsies from the urogenital tract, perianal regions, anal canal, the oral cavity, the upper aerodigestive tract and the epidermis. More preferably, samples are blood, plasma, serum, urine, saliva, or lacrimal fluid. Tissue or organ samples may be obtained from any tissue or organ by, e.g., biopsy or other surgical procedures. Separated cells may be obtained from the body fluids or the tissues or organs by separating techniques such as filtration, centrifugation or cell sorting. It is to be understood that the sample may be further processed in order to carry out the method of the present invention. Particularly, immunoglobulins might be extracted and/or purified from the obtained sample by methods and means known in the art. Thus, the term sample also may relate to antibodies, preferably PV-neutralizing antibodies, purified and/or extracted from any sample as mentioned to above.

The term “contacting” as used in the context of the method of the present invention is understood by the skilled person. Preferably, the term relates to bringing a host cell or sample of the present invention into physical contact with infectious PV particles comprising a reporter gene and thereby allowing said host cell or the compounds comprised in said sample to interact with said PV particles.

The term “PV particle” encompasses viruses (virions) and pseudovirions and is understood by the skilled person. An “infectious PV particle” is a PV particle with the ability to infect a host cell. It is to be understood that not every PV particle in a given population will infect a host cell. In the context of this specification PV particles are assumed to be infectious if at least one particle out of 10, out of 100, out of 1000, out of 10000, or out of 100000 infects a host cell under a given set of conditions. Preferably, infectious PV particles are PV pseudovirions comprising as a genome a polynucleotide comprising at least one non-functional gene essential for PV replication. More preferably, PV particles are PV pseudovirions comprising a capsid consisting of L1 proteins. It is to be understood, however, that the PV pseudovirion may also comprise other polypeptides, e.g. the L2 polypeptide.

In accordance with the present invention, the infectious PV particles shall comprise a reporter gene. Thus the infectious PV particles shall comprise in their genome at least one reporter gene. The term “reporter gene” relates to a gene whose gene product can be detected under appropriate conditions. Preferably, the reporter gene encodes a gene product whose amount or activity can be quantitatively determined. The product of the reporter gene of the present invention is secreted into the growth medium by the host cells, such that the amount or activity of said gene product can be determined without lysing said host cells. More preferably, the reporter gene is encoding the luciferase polypeptide from Gaussia princeps (Genbank Acc. No: AAG54095.1 GI:12621054).

As used herein, a “host cell” is a cell with the ability to be infected by PV maintained in vitro under appropriate conditions. Preferably, the host cell is a mammalian cell, more preferably a human cell. Even more preferably, the host cell is a human cell strongly adhering to a solid support, preferably to multiple cluster plates, and most preferably the host cell is a cell stably transfected with an expression construct for the SV40 large T antigen, e.g. a 293TT or a HeLaT cell (Culp et al. 2006, Papillomavirus Particles Assembled in 293TT Cells Are Infectious In Vivo; J Virol. 80(22): 11381-11384, Kelly et al., 1989, Comeasurement of Simian Virus 40 Early and Late Promoter Activity in HeLa and 293 Cells in the Presence of T Antigen, JOURNAL OF VIROLOGY, Vol. 63, p. 383-391).

“Strongly adhering” in the context of the present invention means the ability of host cells to adhere to a solid support, e.g. multiple cluster plates while plates are handled in a high-throughput facility, meaning the ability to achieve and maintain adhesion to said plates even in the presence of transient translatory and vibratory movement of said plates. It is to be understood that not all cells of a population of strongly adhering cells will adhere to said plates under these conditions; strongly adhering, however, shall mean that at least 40%, at least 50%, at least 60%, at least 70%, at least 80%, or at least 90% of host cells will be adhering to said plates after said conditions are applied.

As used herein “determining the amount of gene product from a reporter gene” relates to determining the amount of gene product generated by the host cells after being infected by the PV particles of the current invention from a reporter gene. Determination is. e.g. achieved by determining the relative amount or the number of molecules of the gene product generated by the host cell, e.g. by immunoblotting. Preferably, determining the amount of the gene product is achieved by measuring a physicochemical property of the gene product, preferably the activity of the gene product produced from the reporter gene, e.g. by the luciferase assay described in Example 4. Other physicochemical properties include, e.g. fluorescence in case of fluorescent proteins, which may be used as reporter gene preferably.

The term “reference amount” is a threshold value used to determine if PV-neutralizing antibodies are present or not. If the amount of PV-neutralizing antibodies determined in a sample exceeds the reference amount, PV-neutralizing antibodies are present; if the amount of PV-neutralizing antibodies determined in a sample is equal or lower than the reference amount, PV-neutralizing antibodies are absent. Likewise, if infection measured as amount or activity of reporter gene product exceeds the reference amount, PV-neutralizing antibodies are absent; if infection measured as amount or activity of reporter gene product is equal or lower than the reference amount, PV-neutralizing antibodies are present. The skilled person knows how to deteiniine the reference amount, e.g. by determining the amount of PV-neutralizing antibodies in a representative set of samples from subjects neither infected with nor immunized against PV and using statistical analysis of the results obtained to deteimine the reference amount. It is to be understood that the reference amount can, preferably, be zero or below the detectable limit of the assay used for determining the reporter gene activity. Thus, the reference amount can be obtained, preferably, from a host cell as defined above which has been contacted to a sample known not to comprise PV-neutralizing antibodies. In such a case, preferably, an amount of reporter gene product obtained by a test sample which is equal or lower than the reference amount will be indicative for a test sample which does not comprise PV-neutralizing antibodies, whereas an amount which is larger than the reference amount is indicative for a test sample comprising PV-neutralizing antibodies.

In another preferred embodiment, the reference amount can be obtained from a host cell as defined above which has been contacted to a sample known to comprise PV-neutralizing antibodies. In such a case, preferably, an amount of reporter gene product obtained by a test sample which is equal or lower than the reference amount will be indicative for a test sample which does comprise PV-neutralizing antibodies, whereas an amount which is larger than the reference amount is indicative for a test sample not comprising PV-neutralizing antibodies.

The definitions made above apply mutatis mutandis to the following:

In a further embodiment, the present invention relates to a host cell strongly adhering to a solid support and, preferably, to multi-cluster plates for use in a method for diagnosing anti-PV immunity comprising the method the current invention.

As used herein, a “method for diagnosing anti-PV immunity” is a method comprising the steps of a) obtaining a suitable sample from a subject, e.g. a serum sample or a mucosal wash sample, b) applying the steps of the method of the current invention to said sample and c) deciding if anti-PV immunity exists in said subject. A “subject” as used herein is a mammal infectible by the PV of the present invention; preferably, the subject is a human. “Immunity” as used herein relates to the ability of a subject's immune system to prevent PV infection from occurring, caused by the presence of T-cells recognizing PV virion epitopes and/or B-cells producing PV-neutralizing antibodies. More preferably, immunity means a long-lasting, most preferably life-long, ability to prevent PV infection from occurring. It will be understood that not all subject in a population will be completely protected from PV infection. However, the term requires that a statistically significant portion of subjects of a cohort or population are effectively prevented from being infected by PV. Whether prevention of infection is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001.

It is to be further understood that anti-PV immunity may be type-specific, meaning e.g. that immunity to HPVI6 does not necessarily mean that the subject's immune system can prevent infection with an other type of PV from occurring.

In a further embodiment, the present invention relates to a device for determination of PV-neutralizing antibodies in a sample comprising a) an analysis unit comprising a host cell strongly adhering to a solid support together with a detector for measuring the amount of gene product of a reporter gene, and b) an evaluation unit comprising a computer implemented algorithm for comparing the detected amount of gene product from the analyzing unit to a stored reference amount.

The term “device” as used herein relates to a system comprising the aforementioned units operatively linked to each other as to allow the diagnosis or monitoring according to the methods of the invention. The term “detector” as used herein refers to an agent which is capable of specifically recognizing the reporter gene product present in a sample. Preferred detection methods are detection of luminescence, fluorescence, or absorbance. The determined amount and/or the presence or the absence of a reporter gene product can be transmitted to the evaluation unit. Said evaluation unit comprises a data processing element, such as a computer, with an implemented algorithm for carrying out a comparison between the determined amount and a suitable reference. Suitable references are either derived from a sample known to comprise PV-neutralizing antibodies or from a sample known not to comprise PV-neutralizing antibodies as described elsewhere herein. The results may be given as output of parametric diagnostic raw data, preferably, as absolute or, more preferably, relative amounts. It is to be understood that these data will need interpretation by the clinician. However, also envisage are expert system devices wherein the output comprises processed diagnostic raw data the interpretation of which does not require a specialized clinician.

In a further embodiment, the present invention relates to a kit for determination of PV-neutralizing antibodies in a sample comprising a host cell as defined in claim 9 or 10.

The term “kit” as used herein refers to a collection of the aforementioned components, preferably, provided in separate or within a single container. The container also comprises instructions for carrying out the method of the present invention. These instructions may be in the form of a manual or may be provided by a computer program code which is capable of carrying out the comparisons referred to in the methods of the present invention and to establish a diagnosis accordingly when implemented on a computer or a data processing device. The computer program code may be provided on a data storage medium or device such as an optical storage medium (e.g., a Compact Disc) or directly on a computer or data processing device.

All references cited in this specification are herewith incorporated by reference with respect to their entire disclosure content and the disclosure content specifically mentioned in this specification.

The following Examples shall merely illustrate the invention. They shall not be construed, whatsoever, to limit the scope of the invention.

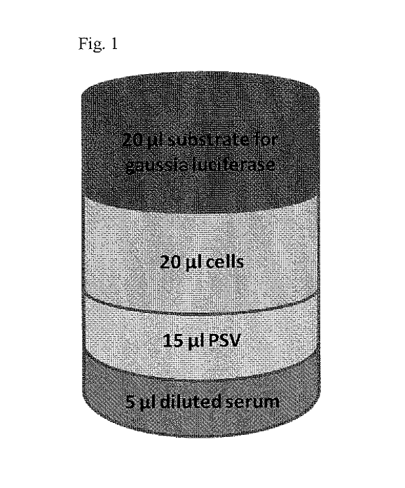

FIG. 1: Overview of pipetting steps in the PBNA

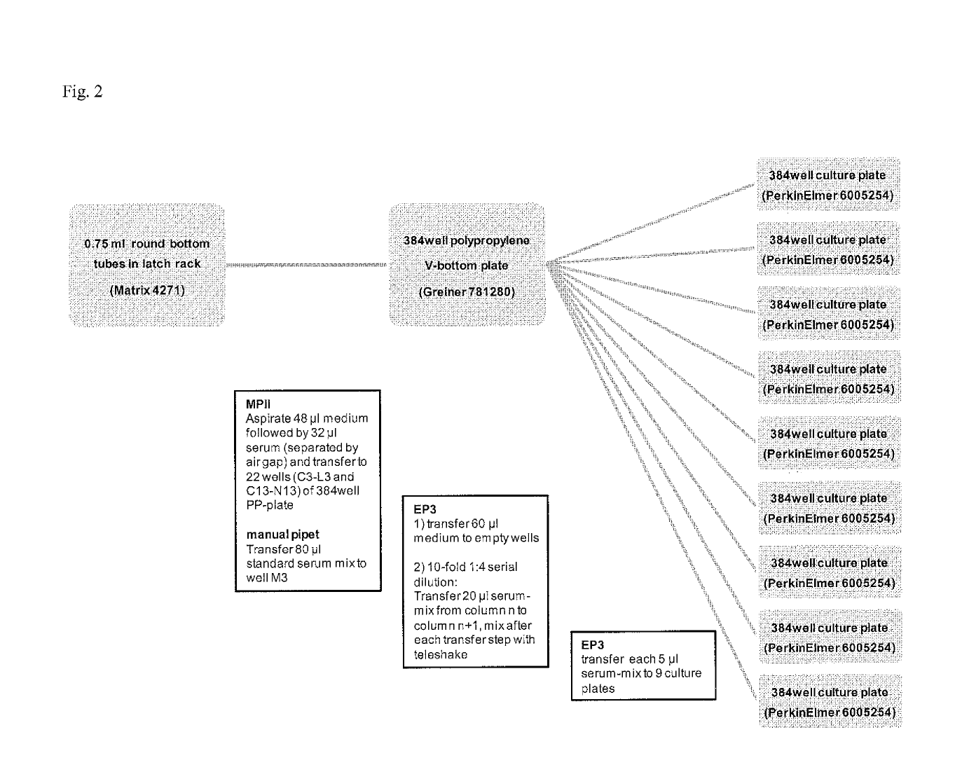

FIG. 2: flow scheme for plate generation

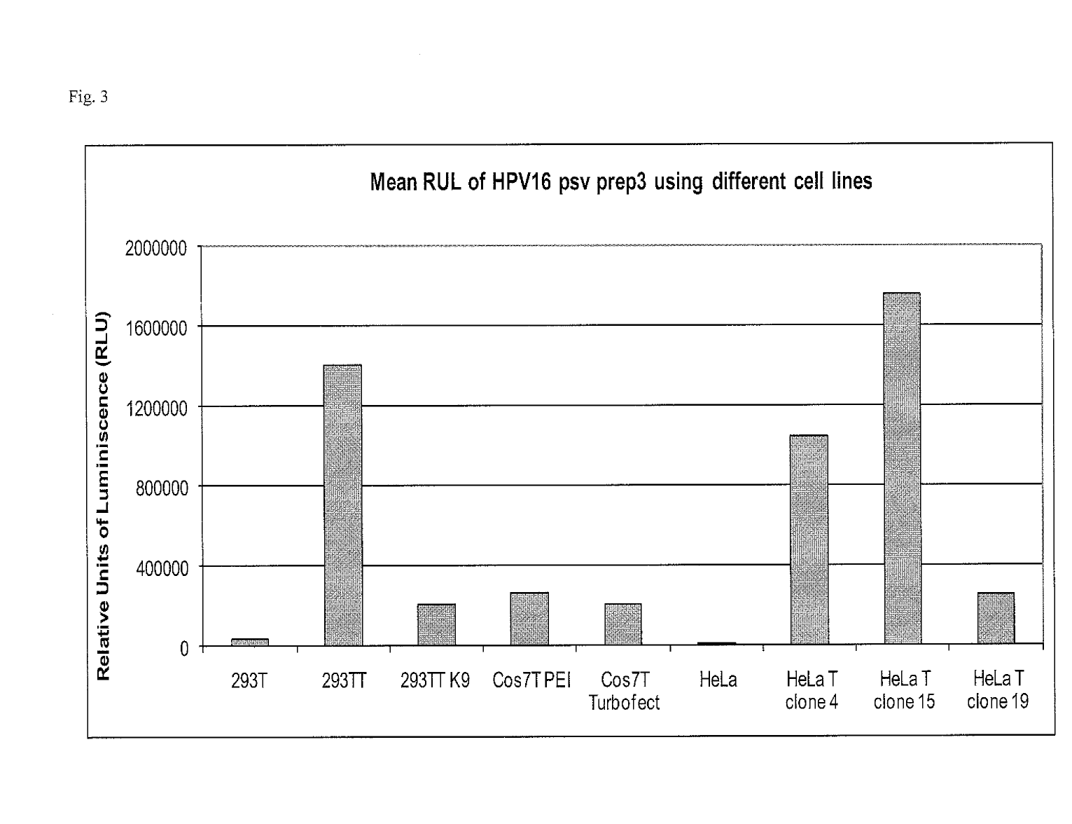

FIG. 3: Mean relative units of luminescence (RLU or RUL) using different cell lines Several cell lines were added to 96well/plates and incubated with HPV16 pseudovirons diluted to a final concentration 1:20,000. Cells were incubated for two days. The assay was developed by transferring 10 μL/well of supernatant to white plates and adding 100 μL/well of Gaussia substrate. Coefficient of variation intra-plate is indicated in the graphic. Values represent the mean of infectivity of 96wells/plate for HPV16 PSV. 293TT and two clones of HeLaT give the higher values.

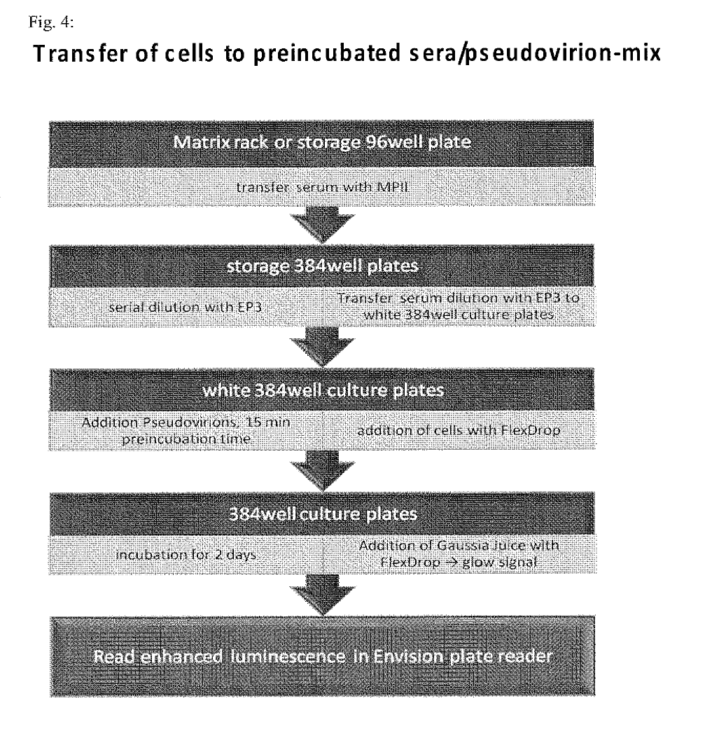

FIG. 4: Scheme of the HiT-PBNA A High Throughput Pseudovirion Based Neutralization assay (HiT-PBNA) is used to detect neutralizing antibodies against Papillomavirus in sample sera. Initially, Sera samples will be pre-diluted with D-MEM and stored in a matrix rack plate, 9 plates will be produced and keep at −20° C. until the assay day. On the day of the assay, matrix plates will be thawed at room temperature and centrifuged, plates will be filled up with pseudovirions and cells using the FlexdropIII as indicated the Examples. Plates will be incubated for 2 days at 37° C. and Gaussia Juice containing coelenterizine (1:100 dilution) will be added to the plates and read using an Envision plate reader.

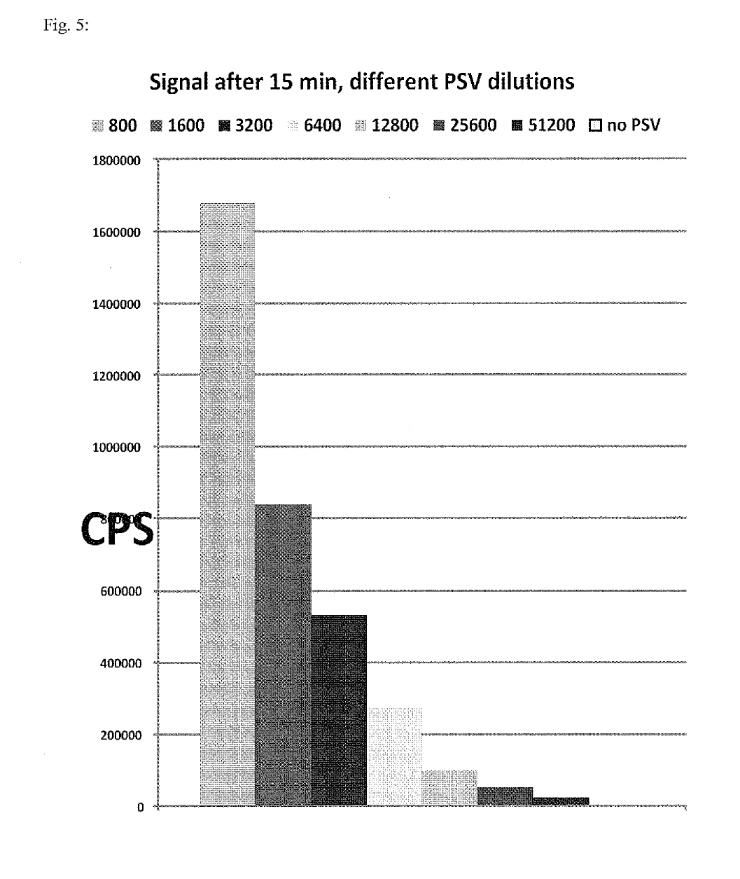

FIG. 5: PSV-concentration dependence of luminescence signal Pseudovirions were added to the plates at the concentrations indicated in the figures. Cells were added to the plates and plates were incubated for two days. After incubation, 20, 30, 40 and 50 μL of Gaussia juice containing coelenterizine (1:100 dilution) were added to the assay to detect the Gaussia activity. There is a proportional relation of PSV-concentration and signal.

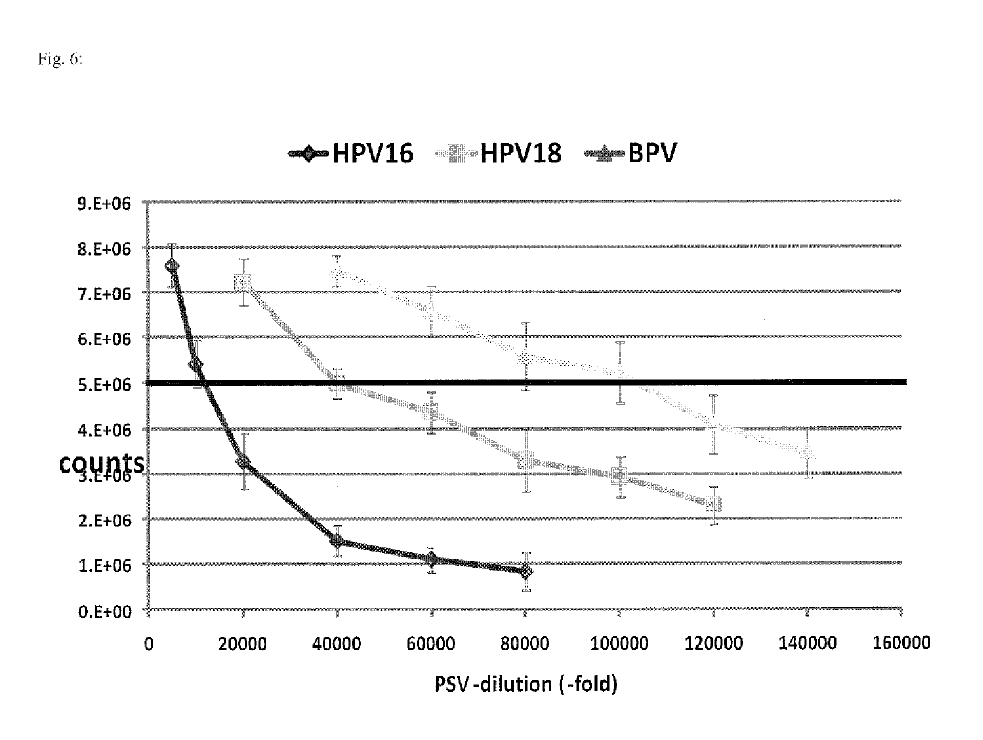

FIG. 6: Adjustment of PSV-dilution for equal Gaussia signal HPV16, HPV18 and BPV1 Pseudovirions were added to the plates at the different concentrations starting from 1:2000 to 1:80000. Cells were added to the plates and incubated for two days. 20 μL of Gaussia juice containing coelenterizine (1:100 dilution) were added to the assay to detect the Gaussia activity. HPV16 at 1:15000, HPV18 at 1:40000 and BPV at 1:100000 gave a similar signal of 5000000 counts.

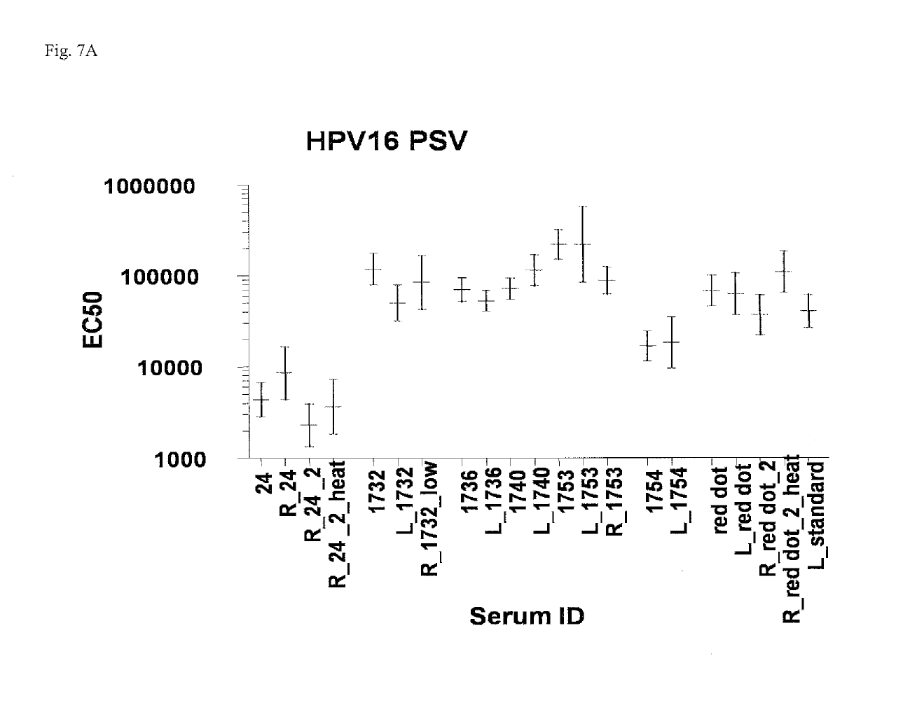

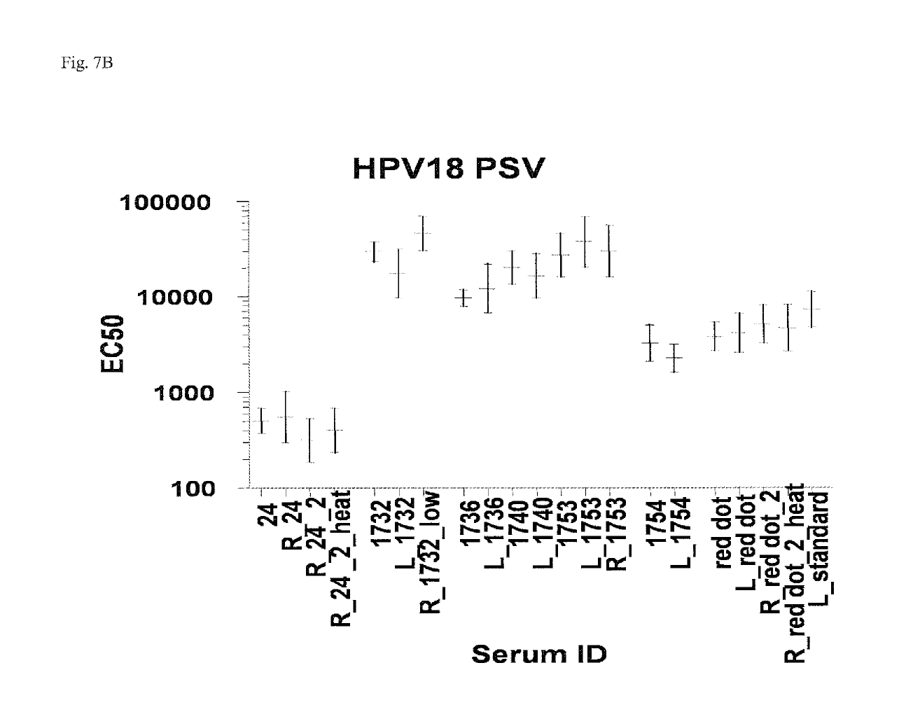

FIG. 7: Interday variability of neutralization titers Three sera of vaccinated woman with high, medium and low antibody reactivity were tested on three different days in the HPV16 and HPV18 neutralizing titration. The experiment was done in triplicates during three different days and is meant to evaluate the interday reproducibility of the neutralizing titer. HPV 16 Pseudovirions (A) were diluted 1:5000 and HPV18 pseudovirions (B) were diluted 1:40000 with DMEM. The cells were added to the plates and plates were incubated for two days. 20 μL of Gaussia juice containing coelenterizine (1:100 dilution) were added to the assay to detect the Gaussia activity. There is a good interday reproducibility of EC50-values.

### EXAMPLES

### Example 1

### Cultivation of Cells

a) Recovering Cells from Liquid Nitrogen Storage

A tube containing 10 ml of supplemented D-MEM medium will be prepared. HeLaT Cells (1.5*10⁶ cells/vial) will be removed from the liquid nitrogen storage and placed into a 37° C. water bath for 1 minute. Once the cells are thawed, they will be transferred to a tube already containing 10 ml of D-MEM supplemented medium.

Cells will be centrifuged for 5 minutes at 1900 rpm at room temperature in a Megafuge 1.0 (Heraeus/SEPATECH) centrifuge. Supernatant will be discarded and the cells will be resuspended in 10 ml supplemented D-MEM medium. Cells will be placed into a 75 cm2 tissue culture flask (TPP) and 10 ml of supplemented medium will be added.

### Cells Will Be Incubated at 37° C.

The growth of the cells will be monitored the next two days. Ideally, cells will form a monolayer and will be confluent in the next six days, but, if the cells start growing forming aggregates, at days 6th after thawing, they will be treated with trypsin, centrifuged and resuspended in 10 ml of fresh medium. All cells will be seed back into the 75 cm2 tissue culture flask to facilitate the formation of the monolayer.

### b) Cell Culture

### aa) Trypsin Treatment of Cells:

Between day 7th to 9th when the cells in the 75 cm2 tissue culture flask are confluent, then the cells will be prepared in big bottles. The amount of big bottles to prepare will depend on the amount of cells needed for the PBNA assay.

To trypsinize the cells, the medium will be aspirated by using a sterile glass Pasteur pipette attached to a vacuum pump.

3 ml of 0.25% Trypsin-EDTA solution in D-MEM medium will be added to the flask and cells will be incubated for 3 minutes at 37° C.

The de-attachment of the cells will be monitored with the microscope and if is still not complete cells will be incubated at 37° C. until they start to detach. The cells will then be resuspended in 10 ml of supplemented medium and centrifuged for 5 minutes at 1900 rpm in a Megafuge 1.0 (Heraeus/SEPATECH) centrifuge.

The supernatant will be discarded and cells will be resuspended in 10 ml of fresh supplemented medium, cells will be counted by using a Neubauer chamber.

### bb) Subcultivation of Cells:

Cells in the 75 cm2 tissue culture flask will be confluent 7 to 9 days after recovering from the nitrogen storage. To maintain the cells in culture, they will be split to a new bottle every 3-4 days.

1.5*10⁶ cells will be added to a 150 cm2 flask together with 30 ml of supplemented D-MEM medium without phenol red and glutamine, Hygromycin (50 mg/ml) will be added at 1:400 dilution. The growth of the cells will be permitted until 90% confluency (reached after 2-3 days).

Cells will be split after 90% confluence, for that, cells will be trypsinized as described above, and 1.5*10⁶ cells will be returned to the flask together with 30 ml of supplemented medium as described before plus Hygromicin diluted 1:400.

Cells will be maintained in culture for up to 4 weeks, after this a new vial of cells will be recovered from the nitrogen storage.

### cc) Preparing Cells for the HiT-PBNA Assay:

For every 10⁷ cells needed, a 150 cm2 flask will be seed with 1.5*10⁶ cells. On the day of the assay, cells will be trypsinized and adjusted to a number 87500 cells/ml.

### Example 2

### High Throughput Pseudovirion Based Neutralization Assay (HiT-PBNA).

### a) Scope

In lack of an animal challenge model, the best system to detect these neutralizing antibodies is a so called Pseudovirion-based neutralization assay. Briefly, Papillomavirus pseudovirions are vehicles consisting of L1 and L2 proteins encapsidating a reporter plasmid. These pseudovirions (PSV) can deliver the DNA to cells in culture, which can be measured by a reporter assay. Delivery can be abrogated in the presence of neutralizing antibodies contained in the serum samples.

In this assay, we use as a reporter the Gaussia Luciferase. This luciferase is secreted to the cell culture supernatants. Gaussia luciferase comes from the marine copepod Gaussia princeps. This luciferase, which does not require ATP, catalyzes the oxidation of the substrate coelenterazine in a reaction that emits light (470 nm). The reaction is read in a Luminometer Evision Plate Reader as will be described below.

This assay is used to detect neutralizing antibodies against human papillomavirus. For this assay, pseudovirions containing the two capsid proteins L1 and L2 from HPV of different types are used to infect HeLa T cells. As a result of the infection, the PSV release in the cell a plasmid encoding for a reporter gene Gaussia Luciferase. Infected cells will be detected by the presence of the luciferase on the culture medium. On the other hand, when neutralizing antibodies are present in the sera, they prevent the infection by pseudovirions and thus expression of the reporter gene. The samples used in this procedure can be serum or plasma from mammal sources (mice, rabbit, human, etc).

### b) Materials

### Pseudovirions

Cells: 293TT cells Human kidney epithelium, 293T transfected and expressing one more copy of large antigen T from SV40 virus

Transfection reagent: Turbofect® (Fermentas)

10% Brij58 in PBS (Sigma)

Benzonase (MERCK)

Optiprep (SIGMA)

### b) Procedure

### aa) Pseudovirion Production and Purification

Pseudovirion production and purification and plasmids used in that procedure have been described before (e.g. Buck et al. 2005, Maturation of Papillomavirus Capsids; Journal of Virology, Vol. 79, No. 5, p. 2839-2846).

### Day 1: Cell Seeding

Prepare a solution of 293TT cells (3*10⁵ cells/ml) in supplemented D-MEM and seed 20 ml per 10 cm dish plate. To make a big preparation of PSV use 10 dishes per PSV.

Day 2: Transfect 293TT Cells Using Turbofect® Like this Per Plate

1 ml of un-supplemented D-MEM

Add 30 μg of total DNA (including plasmids codifying for L1/L2 and Gaussia)

Add 60 μL of Turbofect®

Mix the DNA and the Turbofect® by vortex and incubate at room temperature for 15 minutes

Add the mix to the cells drop by drop

Incubate the cells for three days at 37° C./5% CO2 during 3 to 4 days.

### Pseudovirion Extraction (Day 5 or 6)

Cells are resuspended by pipetting up and down several times the medium, transfer the cell suspension to a 50 ml falcon tube.

Centrifuge the cells at 1900 rpm for 5 minutes, discard the supernatant and re-suspend the cells in 1 ml D-PBS.

Transfer the cells to a 1.5 ml eppendorf tube (low binding tube)

Centrifuge the cells at 5000 rpm for 5 min at 4° C.

Discard the supernatant and re-suspend the cells in equal volume of lyses buffer.

To prepare lyses buffer: 300 μL of DPBS+17.5 μL Brij 58(10%)+2 μL RNaseA/T cocktail

Incubate the cells in lyses buffer for 24 hours at 37° C. under rotation.

### Optiprep Gradient Preparation:

Prepare three initial solutions at 27%, 33% and 39% by dilution of the original solution (60%) using D-PBS/0.8M NaCl to dilute the optiprep. In tubes for SW41TI rotor add 3.2 ml of previous solutions starting with 39% and on top the 27% solution. Leave the tubes overnight at 4° C. to aloud the formation of the gradient.

### Pseudovirion Maturation and Purification (Day 6 or 7)

Place the eppendorf tubes on ice for 5 minutes

Add 0.17 volumes of NaCl 5M to the eppendorf tubes (final concentration 0.8M NaCl in every tube)

Repeat the incubation on ice for 5 minutes

Centrifuge the eppendorf tubes at 10000 rpm for 10 minutes at 4° C.

Collect the supernatants in other eppendorf tube (Eppi # 2) and resuspend the pellets in 300 μL of D-PBS/0.8M NaCl.

Repeat centrifugation at 10000 rpm for 10 minutes at 4° C.

Add the supernatant to the eppendorf tube # 2 (estimated volume of 600 μL)

Add 2 μL of Benzonase and incubate for 1 hour at 37° C.

Centrifuge at 10000 rpm for 10 minutes at 4° C.

Add the supernatant on top of Optiprep gradient (balance the tubes by weight using D-PBS/0.8M NaCl solution)

Centrifuge the gradients at 37000 rpm for 5 hours at 16° C.

Collect 2 ml of fractions starting from lower gradient

Aliquot the purified pseudovirions and freeze at −80° C.

### Example 3

### Serial Dilution of Serum Samples

### a) Equipment and Materials

MultiPROBE® II Plus (MP II) 4-Tip liquid handler; PerkinElmer

Evolution EP3 liquid handling system equipped with 384-well transfer head; PerkinElmer

Teleshake 1536-6 #50101193, Thermo Electron LED GmbH

Semi-automated plate sealer RoboSeal, #760B, HJ-Bioanalytik

### c) Procedure

flow scheme for plate generation: FIG. 2

Labeling of Plate with Barcodes

Stick barcodes to the short right side of assay plates and 384well PP-plate for serial dilution of the sera.

### B. Serum Dilution

Thaw sera and centrifuge latch rack for 1 min at 1.000 g in Heraeus Megafuge 1.0 R

With MPII robot: Aspirate 48 μl medium followed by 32 μl serum (separated by air gap) and transfer to 22 wells (C3-L3 and C13-N13) of 384well PP-plate

With manual pipet: Transfer 80 μl standard serum mix to well M3

With EP3: Add 60 μl medium to empty wells and prepare a 10-fold 1:4 serial dilution by transferring 20 μl serum-mix from column n to column n+1. Mix after each transfer step with teleshake (1400 rpm, 45 sec)

### B. Assay Plate Preparation

With EP3: Transfer each 5 μl serial diluted serum from 384well PP-plate to 9 assay plates (white 384well culture plate)

Immediately seal assay plates with PP cover foil and store at −20° C.

### Example 4

### High Throughput Pseudovirion Based Neutralization Assay

### a) Introduction

A High Throughput Pseudovirion Based Neutralization assay (HiT-PBNA) is used to detect neutralizing antibodies against Papillomavirus in sample sera. Papillomavirus pseudovirions (PSV) are vehicles consisting of L1 and L2 proteins encapsidating a reporter plasmid. Transduction of the cells and thereby expression of the reporter can be abrogated by neutralizing antibodies contained in the serum samples.

The luciferase from the marine copepod Gaussia princeps is used as reporter in this assay.

Gaussia luciferase is secreted to the cell culture supernatant and does not require ATP for the oxidation of its substrate coelenterazine. The luminescence originating from this reaction is measured in a Plate Reader.

### b) Equipment and Materials

### Equipment

FlexDropIV™ Reagent Dispenser, PerkinElmer

Envision 2101 Multilabel Reader; PerkinElmer

### Procedure

Overview of pipetting steps in the PBNA: FIG. 1

### Assay Assembly

Thaw assay plates (see Example 3), centrifuge for 1 min at 1.000 g in Heraeus Megafuge 1.0 R and remove cover foil

With FlexDrop: Add 15 μl/well PSV diluted in medium to assay plates, incubate for 15 min.

The final dilution of the PSV in the assay after adding the cells are: HPV16 at 1:15000, HPV18 at 1:40000 and BPV at 1:100000

With FlexDrop: Add 20 μl/well medium to one 384well clear culture plate for the control of cells by microscopy

With FlexDrop. Add 20 μl/well HeLaT K4 cells (87500 cells/ml→1750 cells/well) to all plates. Bring plates to incubator and incubate for 2 days at 37° C./%5 CO2

Check cells in the 384well clear control plate by microscopy directly after seeding and then once per day

### Read Out

Remove plates from the incubator and equilibrate them to RT

Dilute the substrate Coelenterazine 1:100 in substrate buffer (10 mg/l coelenterazine in “gaussia glow juice”)

With FlexDrop: Add 20 μl/well substrate solution. Place a batch of 9 plates into the right stacker of the Envision 2101 plate reader. The reader is equipped with an internal bar code reader.

Measure the luminescence (0.1 sec/well). Results of each plate are separately stored as text file in matrix format.

1-13. (canceled)

14. A method for the determination of PV-neutralizing antibodies in a sample comprising:
(a) contacting the sample with infectious PV particles comprising a reporter gene, wherein the gene product of the reporter gene is secreted into the growth medium;
(b) contacting the PV particles from (a) with host cells; and
(c) determining PV-neutralizing antibodies based on the amount of gene product from the reporter gene.

15. The method of claim 14, wherein a lower amount of the gene product as compared to a reference amount in step (c) is indicative of the presence of PV-neutralizing antibodies.

16. The method of claim 14, wherein the determination is a quantitative determination.

17. The method of claim 14, wherein the infectious PV particles are PV pseudovirions.

18. The method of claim 14, wherein the host cells are cells strongly adhering to a solid support, preferably a multi-cluster plates.

19. The method of claim 14, wherein the host cells are HeLa-T cells.

20. The method of claim 14, wherein all steps of the method are performed in the same multicluster plate.

21. The method of claim 14, wherein the product of the reporter gene is Gaussia luciferase.

22. A host cell strongly adhering to a solid support for use in a method for diagnosing anti-PV immunity according to the method of claim 14.

23. The host cell of claim 22, wherein the host cell is a HeLa-T cell.

24. A device for determination of PV-neutralizing antibodies in a sample comprising
(a) an analysis unit comprising a host cell strongly adhering to a solid support together with a detector for measuring the amount of gene product of a reporter gene; and
(b) an evaluation unit comprising a computer implemented algorithm for comparing the detected amount of gene product from the analyzing unit to a stored reference amount.

25. A kit for determination of PV-neutralizing antibodies in a sample comprising a host cell as defined in claim 22.
